(19) United States
(12) Patent Application Publication
(10) Pub. No.: US 2025/0241803 A1
(43) Pub. Date: Jul. 31, 2025

(54) FEMININE HYGIENE PRODUCT INCLUDING A CRIMPED REGION AND ASSOCIATED METHOD

(71) Applicant: The Procter & Gamble Company, Cincinnati, OH (US)

(72) Inventors: Christian KLEBBA, SCHWALBACH (DE); Alexander ZERBOCK, Bad Soden am Taunus (DE)

(21) Appl. No.: 19/032,527

(22) Filed: Jan. 21, 2025

Related U.S. Application Data
(60) Provisional application No. 63/624,829, filed on Jan. 25, 2024.

(51) Int. Cl. A61F 13/56; A61F 13/15; A61F 13/515

(52) U.S. Cl. CPC A61F 13/5616; A61F 13/1565; A61F 13/15739; A61F 13/515; A61F 2013/15715

(57) ABSTRACT

A feminine hygiene article is provided with a topsheet, a backsheet and an absorbent core between the topsheet and backsheet. The absorbent core has first and second longitudinal core edges. The feminine hygiene article includes a first wing extending laterally outboard the first longitudinal core edge. The first wing includes a first layer, a second layer, a leading edge portion, a trailing edge portion and an outer most edge portion. The first wing has a first wing portion extending from the first longitudinal core edge to a first edge and a second wing portion extending from the first edge to the outer most edge portion. The first and second layers are joined by an adhesive in the first wing portion. The first layer and the second layer are joined by mechanical bonding in the second wing portion. A method is also provided for forming the improved feminine hygiene product.

### CROSS REFERENCE TO RELATED APPLICATIONS

This application claims the benefit, under 35 U.S.C. § 119 (e), to U.S. Provisional Application No. 63/624,829, filed Jan. 25, 2024, the entire disclosure of which is fully incorporated by reference herein.

### FIELD

The present disclosure relates generally to absorbent articles such as feminine hygiene products and more specifically to an improved feminine hygiene product with enhanced production efficiency.

### BACKGROUND

All manner and variety of absorbent articles configured for the absorption of body fluids such as menses, urine, and feces are, of course, well known. Absorbent articles, particularly feminine hygiene products having wings or flaps are disclosed in the literature and are available in the marketplace.

Generally, the wings extend laterally from a central absorbent means and are intended to be folded around the edges of the wearer's panties in the crotch region. Thus, the wings are disposed between the edges of the wearer's panties in the crotch region and the wearer's thighs. Commonly, the wings are provided with an attachment means for affixing the flaps to the underside of the wearer's panties.

As shown in FIG. 1A, conventional feminine hygiene products 10 with wings are manufactured using a process that features a top sheet 102 having a cut width 160 that exceeds a final width 164 of the manufactured feminine hygiene product 10. Additionally, as shown in FIG. 1A, the conventional manufacturing method uses adhesive 126 with a width 162 which also exceeds the final width 164 of the manufactured feminine hygiene product 10. Thus, such conventional methods necessarily cut away a significant amount of excess material including the top sheet 102 and the adhesive 126, leading to a significant amount of material being wasted.

The discussion of shortcomings and needs existing in the field prior to the present disclosure is in no way an admission that such shortcomings and needs were recognized by those skilled in the art prior to the present disclosure.

### SUMMARY

Various aspects solve the above-mentioned problems and provide methods and devices useful for addressing this issue of significant material waste in a cost-effective and environmentally friendly manner. Specifically, as shown in FIG. 1B, the aspects of the disclosure propose reducing the cut width 160′ of the top sheet 102 as compared with the cut width 160 of the top sheet 102 in the conventional method. Additionally, as shown in FIG. 1B, the aspects of the disclosure also propose reducing the cut width 162′ of the adhesive 126 that is used in method as compared to the increased cut width 162 of adhesive 126 in the conventional method. Indeed, by simply decreasing the width of the starting material, such as the widths 160′, 162′ of the top sheet 102 and adhesive 126, not only is the amount of waste minimized.

It was recognized that variations in the manufacturing process are unavoidable and thus reducing the initial widths of these starting materials may introduce other problems, such as potential exposure of the underling glue or adhesive 126 in the wing area of the feminine hygiene product. Thus, various aspects of the disclosure are provided to thwart this potential problem so that reducing the widths of the starting material does not introduce this unwanted outcome. In one aspect of the disclosure, to address the issue of adhesive exposure, one solution is to reduce the dimension of the adhesive 126 in the wing area and ensure the closure of the wing by introducing a mechanical bonding 128 region (e.g., crimped area). This solution offers several advantages. Firstly, it permits use of materials (e.g., adhesive 126 and topsheet 102) with a reduced cut width, thereby minimizing waste. Secondly, it effectively prevents glue or adhesive 126 exposure, ensuring that the topsheet 102 remains free from contamination. Lastly, this solution maintains the integrity of the feminine hygiene product 100, ensuring its quality and functionality.

In a first set of aspects of the disclosure, a feminine hygiene article is provided that includes a topshect, a backsheet and an absorbent core disposed between the topsheet and the backsheet. The absorbent core has a first longitudinal core edge and a second longitudinal core edge. The feminine hygiene article also includes a first wing extending laterally outboard the first longitudinal core edge. The first wing includes a first layer, a second layer, a leading edge portion, a trailing edge portion and an outer most edge portion joining the leading edge portion and the trailing edge portion. The first wing has a first wing portion extending from the first longitudinal core edge to a first edge and a second wing portion extending from the first edge to the outer most edge portion. The first layer and the second layer are joined by an adhesive in the first wing portion. The first layer and the second layer are joined by mechanical bonding in the second wing portion.

In a second set of aspects of the disclosure, a feminine hygiene article is provided that includes a topsheet, a backsheet and an absorbent core disposed between the topsheet and the backsheet. The absorbent core has a first longitudinal core edge and a second longitudinal core edge. The feminine hygiene product also includes a first wing extending laterally outboard the first longitudinal core edge. The first wing includes a portion of the topsheet, a portion of the backsheet, a leading edge portion, a trailing edge portion and an outer most edge portion joining the leading edge portion and the trailing edge portion. The first wing has a first wing portion extending from the first longitudinal core edge to a first edge and a second wing portion extending from the first edge to the outer most edge portion. The first layer and the second layer are joined by an adhesive in the first wing portion. The first layer and the second layer are joined by mechanical bonding in the second wing portion. An overlap is provided between the adhesive of the first wing portion and the mechanical bonding of the second wing portion, where the overlap is greater than 0 mm. The first wing has a wearer facing surface and a garment facing surface, where the garment facing surface comprises a garment fastening adhesive. A gap is present between the garment facing adhesive and the second wing portion, where the gap is greater than 0 mm.

In a third set of aspects of the disclosure, a method is provided for forming an absorbent article or feminine hygiene article. The method includes providing a topsheet web and advancing the topsheet web in a machine direction. The topsheet web has a topsheet width measured in a direction parallel to a cross direction that is perpendicular to the machine direction. The method also includes providing a core material and depositing the core material onto a first surface of the topsheet web material. The method also includes providing a backsheet web. The method also includes advancing the backsheet web in the machine direction and depositing the backsheet web onto the topsheet web such that the first surface of the topsheet web is in a facing relationship with a first surface of the backsheet web. The backsheet has a backsheet width measured in a direction parallel to the cross direction, where the topsheet width is less than the backsheet width. The method also includes applying adhesive between the backsheet web and the topsheet web in a first wing and a second wing. The adhesive is deposited in only a portion of each of the first wing and the second wing. The method also includes crimping an outer portion of at least one of the first wing and the second wing.

These and other features, aspects, and advantages of various embodiments will become better understood with reference to the following description, figures, and claims.

### BRIEF DESCRIPTION OF THE DRAWINGS

Many aspects of this disclosure can be better understood with reference to the following figures, which illustrate examples according to various embodiments.

FIG. 1A is an example illustrating a top view of topsheet material and adhesive material used to manufacture a feminine hygiene product with a conventional manufacturing process;

FIG. 1B is an example according to various aspects illustrating a top view of a reduced width of topsheet material and adhesive material used to manufacture a feminine hygiene product in an improved manufacturing process;

FIG. 2 is an example according to various aspects illustrating a top view of a feminine hygiene product with asymmetrical wings formed using the improved manufacturing process;

FIG. 3 is an example according to various aspects illustrating a cross-sectional view of the feminine hygiene product taken along the line 3-3 in FIG. 2;

FIG. 4A is an example according to various aspects illustrating a cross-sectional view of the feminine hygiene product taken along the line 4-4 in FIG. 2;

FIG. 4B is an example according to various aspects illustrating a cross-sectional view of the feminine hygiene product taken along the line 4-4 in FIG. 2;

FIGS. 5A and 5B are examples according to various aspects illustrating a top view of a feminine hygiene product with symmetrical wings formed using the improved manufacturing process;

FIG. 6A is an example according to various aspects illustrating a top view of the feminine hygiene product of FIG. 2 with the adhesive layers and mechanical bonding region formed using the improved manufacturing process;

FIG. 6B is an example according to various aspects illustrating a top view of the first wing of the feminine hygiene product of FIG. 6A including first and second wing portions;

FIG. 6C is an example according to various aspects illustrating a top view of the first wing of the feminine hygiene product of FIG. 6A including the adhesive layers and mechanical bonding region in the first and second wing portions;

FIG. 7 is an example according to various aspects illustrating a flow chart depicting steps of an improved method for forming the feminine hygiene product of FIG. 2; and

FIGS. 8A through 8E are examples according to various aspects illustrating block diagrams that depict the performance of different steps of the method of FIG. 7.

It should be understood that the various embodiments are not limited to the examples illustrated in the figures.

### DETAILED DESCRIPTION

### Introduction and Definitions

This disclosure is written to describe the invention to a person having ordinary skill in the art, who will understand that this disclosure is not limited to the specific examples or embodiments described. The examples and embodiments are single instances of the invention which will make a much larger scope apparent to the person having ordinary skill in the art. Unless defined otherwise, all technical and scientific terms used herein have the same meaning as commonly understood by the person having ordinary skill in the art. It is also to be understood that the terminology used herein is for the purpose of describing examples and embodiments only, and is not intended to be limiting, since the scope of the present disclosure will be limited only by the appended claims.

All the features disclosed in this specification (including any accompanying claims, abstract, and drawings) may be replaced by alternative features serving the same, equivalent, or similar purpose, unless expressly stated otherwise. Thus, unless expressly stated otherwise, each feature disclosed is one example only of a generic series of equivalent or similar features. The examples and embodiments described herein are for illustrative purposes only and that various modifications or changes in light thereof will be suggested to the person having ordinary skill in the art and are to be included within the spirit and purview of this application. Many variations and modifications may be made to the embodiments of the disclosure without departing substantially from the spirit and principles of the disclosure. All such modifications and variations are intended to be included herein within the scope of this disclosure. For example, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All numeric values are herein assumed to be modified by the term “about,” whether or not explicitly indicated. The term “about” generally refers to a range of numbers that one of skill in the art would consider equivalent to the recited value (for example, having the same function or result). In many instances, the term “about” may include numbers that are rounded to the nearest significant figure.

In everyday usage, indefinite articles (like “a” or “an”) precede countable nouns and noncountable nouns almost never take indefinite articles. It must be noted, therefore, that, as used in this specification and in the claims that follow, the singular forms “a,” “an,” and “the” include plural referents unless the context clearly dictates otherwise. Thus, for example, reference to “a support” includes a plurality of supports. Particularly when a single countable noun is listed as an element in a claim, this specification will generally use a phrase such as “a single.” For example, “a single support.”

Unless otherwise specified, all percentages indicating the amount of a component in a composition represent a percent by weight of the component based on the total weight of the composition.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

In this specification and in the claims that follow, reference will be made to a number of terms that shall be defined to have the following meanings unless a contrary intention is apparent.

“Disposed on” refers to a positional state indicating that one object or material is arranged in a position adjacent to the position of another object or material. The term does not require or exclude the presence of intervening objects, materials, or layers.

“Absorbent article” refers to devices that absorb and contain liquid, and more specifically, refers to devices that are placed against or in proximity to the body of the wearer to absorb and to contain various exudates discharged from the body.

“Align” or “aligned” or “aligning” means to place or to arrange in a substantially straight line. Aligning edges of substrates, therefore, means arranging the substrates so that the edges in question extend along approximately the same line. It is to be appreciated that aligning edges of substrates can be accomplished in a variety of ways, including placing the substrates one on top of the other or side by side.

“Facing relationship” refers to a relative positioning of materials, such as substrates, in which a surface of one material is oriented toward a surface of another material. For example, when two substrates are stacked on top of each other, they are in a facing relationship. The term does not require or exclude the presence of intervening objects, materials, or layers.

“Machine direction” (MD) refers to the direction of material flow through a process. In addition, relative placement and movement of material can be described as flowing in the machine direction through a process from upstream in the process to downstream in the process.

“Cross direction” (CD) refers to a direction that is generally perpendicular to the machine direction.

### Feminine Hygiene Product

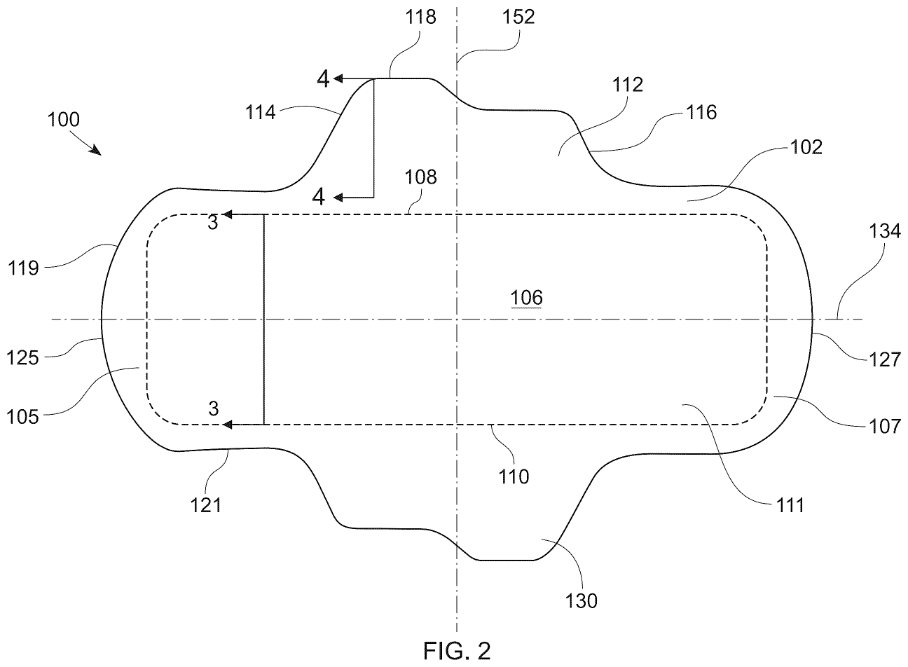

FIG. 2 is an example according to various aspects illustrating a top view of a feminine hygiene product 100 with asymmetrical wings 112, 130 formed using the improved manufacturing process. As shown in FIG. 2, the feminine hygiene product 100 comprises an absorbent means represented by central absorbent pad (or “main body portion”) 111, and two wings 112, 130. In the discussion that follows, unless otherwise noted, the feminine hygiene product described herein will have two wings. While it is not necessary that the feminine hygiene product have two wings, two wings are preferred over one wing. Also, while it is not necessary that the wings be mirror images of one another, they preferably are mirror images of one another. Thus, the description of one wing will be a description of the other, and, for clarity, discussion of the second wing may be omitted.

The feminine hygiene product 100 has two centerlines, a principal longitudinal centerline 134 and a principal transverse centerline or transverse wing axis 152. The term “longitudinal”, as used herein, refers to a line, axis or direction in the plane of the feminine hygiene product 100 that is generally aligned with (e.g., approximately parallel to) a vertical plane which bisects a standing wearer into left and right body halves when the feminine hygiene product 100 is worn. The terms “transverse” or “lateral” used herein, are interchangeable, and refer to a line, axis or direction which lies within the plane of the feminine hygiene product 100 that is generally perpendicular to the longitudinal direction.

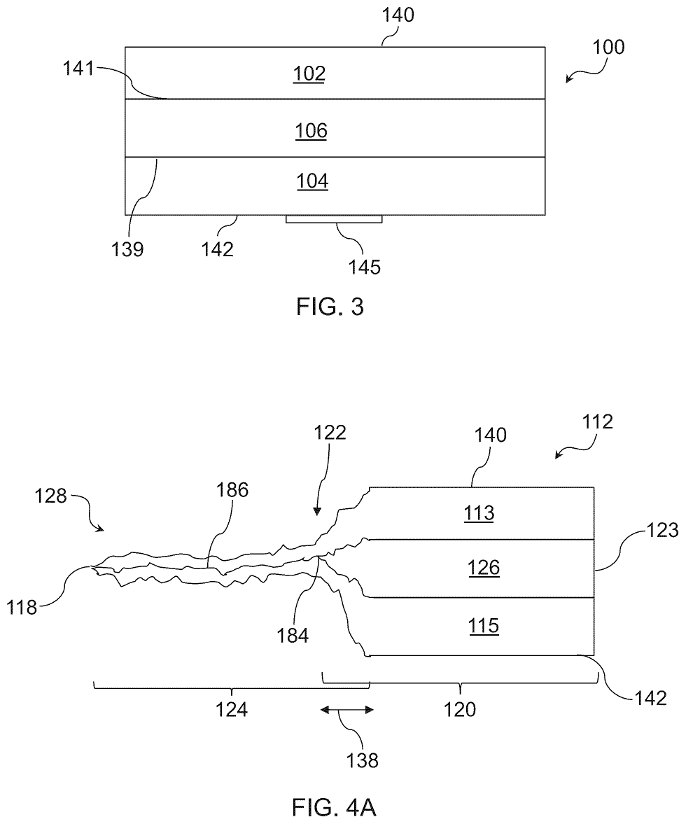

Referring now to FIGS. 2 and 3, the feminine hygiene product 100 is comprised of a topsheet 102, a backsheet 104, an absorbent core 106, and a pair of wings 112, 130. At least a part of the topsheet 102, backsheet 104, and absorbent core 106 comprise the main body portion 111. The topsheet 102 is liquid permeable and when the feminine hygiene product 100 is in use, the topshect 102 is in close proximity to the skin of the user. The topsheet 102 is compliant, soft feeling, and non-irritating to the user's skin. It can be made from any of the materials conventional for this type of use. Nonlimiting examples of suitable materials that can be used as topsheet 102 are woven and nonwoven cellulosic fibers (such as cotton, flax, hemp, jute, and kenaf), polyester, polypropylene, nylon, and rayon and formed thermoplastic films, with formed films being preferred.

Suitable formed films are described in U.S. Pat. No. 3,929,135, entitled “Absorptive Structure Having Tapered Capillaries”, which issued to Thompson on Dec. 30, 1975, U.S. Pat. No. 4,324,426, entitled “Disposable Absorbent Article Having A Stain-Resistant Topsheet”, which issued to Mullane and Smith on Apr. 13, 1982, U.S. Pat. No. 4,342,314, entitled “Resilient Plastic Web Exhibiting Fiber-Like Properties”, which issued to Radel and Thompson on Aug. 3, 1982, and U.S. Pat. No. 4,463,045, entitled “Macroscopically Expanded Three-Dimensional Plastic Web Exhibiting Non-Glossy Visible Surface and Cloth-Like Tactile Impression”, which issued to Ahr, Louis, Mullane, and Ouellette on Jul. 31, 1984.

In addition, in some aspects of the disclosure, at least a portion of an outer surface (wearer facing surface 140 in FIG. 3) of the topsheet 102 is treated with a surfactant. It is preferred that the surfactant be substantially evenly and completely distributed across at least the portion of the outer surface of the topsheet 102 that overlays the main body portion 111. This can be accomplished by any of the common techniques well known to those skilled in the art. For example, the surfactant can be applied to topsheet 102 by spraying, by padding, or by the use of transfer rolls.

Treating the outer surface (wearer facing surface 140 in FIG. 3) of the topsheet 102 with a surfactant renders the surface of the topsheet 102 more hydrophilic. This results in liquid penetrating the topsheet 102 faster than it would if the surface were not treated. This diminishes the likelihood that menstrual fluids will flow off topsheet 102 rather than being absorbed by the absorbent core 106. Preferably, any portions of the topsheet 102 that overlay the wings 112, 130 are not treated with the surfactant. This will minimize any tendencies fluids may have to spread laterally across the wings and to come in contact with the wearer's thighs and other parts of the wearer's body.

In some aspects, an inner surface 141 (FIG. 3) of the topsheet 102 is secured in contacting relation with the absorbent core 106. This contacting relationship results in liquid penetrating the topsheet 102 faster than if the topsheet 102 were not in contact with the absorbent core 106. The topsheet 102 can be maintained in contact with the absorbent core 106 by applying adhesive to the inner surface 141 of the topsheet 102. Suitable adhesives useful for this purpose are described in U.S. Pat. No. 4,917,697. The adhesives can be applied by the same methods as the surfactant is applied to the outer surface (wearer facing surface 140) of the topsheet 102.

As shown in FIG. 3, the absorbent core 106 is positioned between the topsheet 102 and the backsheet 104. The absorbent core 106 provides the means for absorbing menstrual fluid. The absorbent core 106 need not have an absorbent capacity much greater than the total amount of menstrual fluid anticipated to be absorbed. The absorbent core 106 is generally compressible, conformable, and non-irritating to the user's skin. It can comprise any material used in the art for such purpose. Examples include comminuted wood pulp which is generally referred to as airfelt, creped cellulose wadding, absorbent foams, absorbent sponges, synthetic staple fibers, polymeric fibers, hydrogel-forming polymer gelling agents, peat moss, or any equivalent material or combinations of materials.

Polymeric gelling agents are those materials which, upon contact with fluids (i.e., liquids) such as water or body fluids, imbibe such fluids and thereby form hydrogels. In this manner, fluids discharged into the absorbent core 106 can be acquired and held by the polymeric gelling agent, thereby providing the articles herein with enhanced absorbent capacity and/or improved fluid retention performance.

The polymeric gelling agent which is employed in the absorbent core 106 will generally comprise particles of a substantially water-insoluble, slightly cross-linked, partially neutralized, hydrogel-forming polymer material. The term “particles”, as used herein, can refer to particles in any form, such as in the form of pellets, flakes, or fibers. The characteristics of the absorbent core 106 (including, but not limited to the preferred types of polymer materials used therein, and types of methods which can be used for preparing these polymer particles) are described in greater detail in U.S. Pat. No. 5,009,653 issued to Osborn and the patents incorporated by reference in that patent.

In one aspect, the absorbent core 106 is a laminate comprised of a layer of superabsorbent polymer material, such as in the form of particles, disposed between two air-laid tissues, first and second tissue layers (or “upper” and “lower” tissue layers). The first and second tissue layers provide containment of the superabsorbent polymer material, improve lateral wicking of the absorbed exudates throughout the absorbent core 106 and provide a degree of absorbency.

The backsheet 104 is impervious to liquids and, thus, prevents menstrual fluid from soiling the clothing of the user. Any material used in the art for such purpose can be utilized herein. Suitable materials include embossed or nonembossed polyethylene films and laminated tissue.

In one aspect of the feminine hygiene product 100 (typically in which the topsheet 102 overlays only the main body portion 111 and does not extend out to form the top surface of the wings), the backsheet 104 may be comprised of two layers. In such a case, the backsheet 104 may comprise a first layer of lofted material disposed on the core-facing side 139 of the backsheet 104. The purpose of the first layer is to provide a comfortable, non-irritating surface against the body of the wearer. The lofted layer may be comprised of any suitable material, such as a nonwoven material. Preferably, the lofted layer comprises a hydrophobic nonwoven material. The second layer may be disposed on the garment facing surface 142 of the backsheet 104, and may comprise a fluid impervious film. A low density polyethylene material about 0.01 to about 0.05 millimeters in thickness, preferably about 0.02 millimeters in thickness, has been found to work well as this second layer. A polyethylene film, such as is sold by the Ethyl Corporation, Visqueen Division, under model XP-39385 has been found particularly well suited for this second layer. The backsheet 104 may also be made of a soft, cloth-like material which is hydrophobic relative to the topsheet 102. A polyester or polyolefinic fiber backsheet 104 may also be used. A particularly preferred soft, cloth-like backsheet 104 material is a laminate of a polyester nonwoven material and a film such as described in U.S. Pat. No. 4,476,180 issued to Wnuk on Oct. 9, 1984.

The topsheet 102 is joined or secured to backsheet 104 along a seam 119. The seam 119 can be formed by any means commonly used in the art for this purpose such as by gluing, crimping, or heat-scaling. The topsheet 102 may also be indirectly joined to the backsheet 104.

The main body portion 111 is the portion of the feminine hygiene product 100 that contains an absorbent means, such as absorbent core 106. The main body portion 111 has a liquid pervious body contacting surface and an opposed liquid impervious surface. It is to be understood that the aspect illustrated is only one possible aspect, albeit a preferred one. Other possible aspects include one in which an absorbent core 106 is essentially completely wrapped with the topsheet before it is placed on the backsheet. The main body portion 111 can also comprise an absorbent core which possesses sufficient integrity to stand alone and is liquid pervious on one surface while the other surface has been treated to render it liquid impervious.

The main body portion 111 may be relatively thick or relatively narrow and thin. The main body portion 111 may be configured to maintain a position in relatively close proximity to the body. Such proximity of main body portion 111 places it precisely where it should be: near the body at the vaginal opening. The main body portion 111 can then absorb the vast majority of the menstrual fluid (menses) before the fluid has an opportunity to flow along the sides of the main body portion 111. Additionally, a relatively thin main body portion may be desired because it is typically more comfortable to the user.

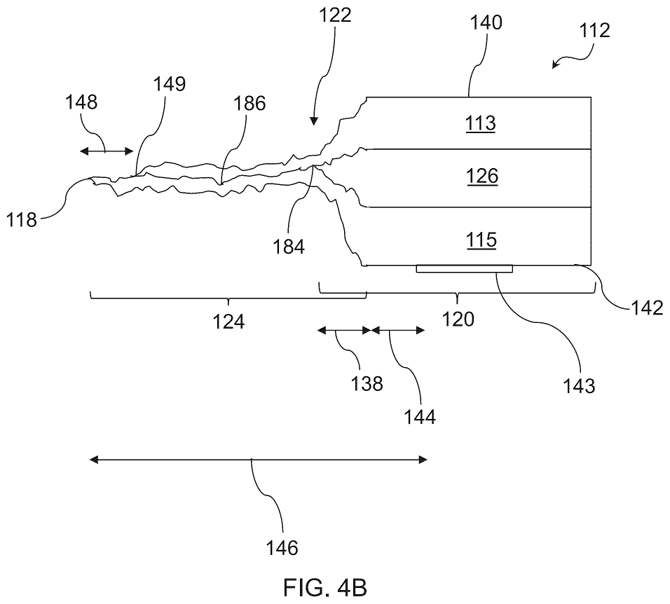

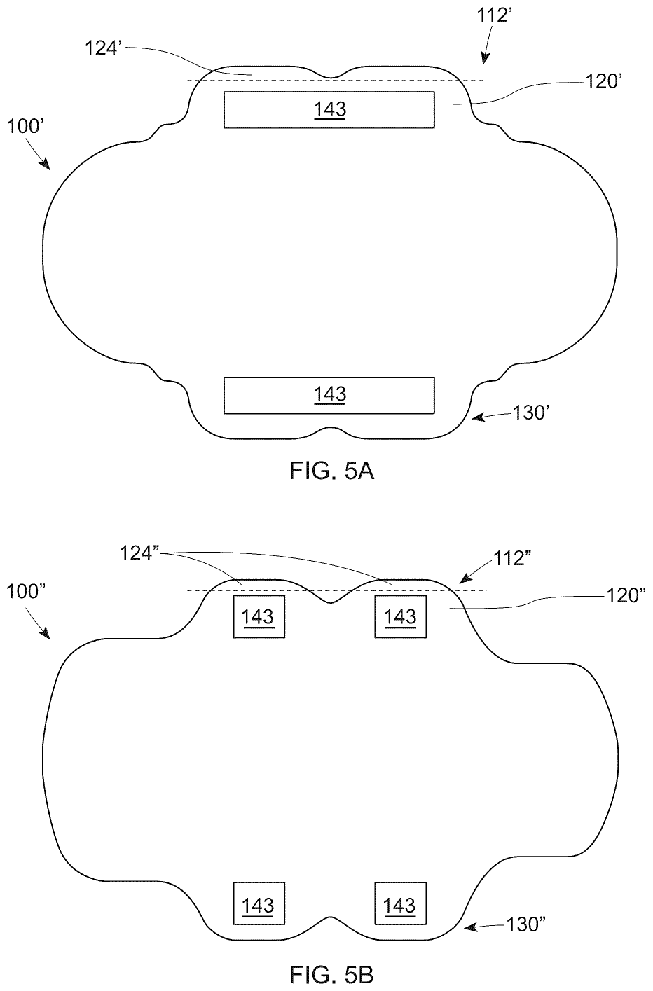

Fasteners, such as adhesive attachment means, central pad adhesive 145 (FIG. 3) and garment facing adhesive 143 (FIG. 4B), are provided to secure the feminine hygiene product 100 to the crotch region of an undergarment.

The central pad adhesive 145 provides an adhesive attachment means for securing main body portion 111 in the crotch portion of a panty. The outer surface of wing 112 includes the garment facing adhesive 143 (FIG. 4B). The garment facing adhesive 143 is used to assist in maintaining the wing 112 in position after it is wrapped around the edge of the crotch portion of the panty as described below. The wings 112, 130 can be maintained in position by attaching the wings 112, 130 to the undergarment, or to the opposing flap. Suitable adhesive fasteners are described in greater detail in U.S. Pat. No. 4,917,697.

Each wing 112, 130 comprises a pair of garment facing adhesives 143. In some aspects, as shown in FIG. 4B the garment facing adhesive 143 is provided in a first wing portion 120 of the first wing 112 that is inside a second wing portion 124 that extends to the outer most edge portion 118 of the wing 112. While the aspect of FIG. 4B depicts a single garment facing adhesive 143 in the first wing portion 120, in other aspects more than one garment facing adhesive 143 is provided and can be positioned in regions of the wing 112 other than the first wing portion 120, such as in the second wing portion 124.

The fasteners used with the disclosure herein are not limited to adhesive attachment means. Any type of fastener used in the art can be used for such purpose. For example, the feminine hygiene product 100 could be secured to the wearer's undergarment by the fastener described in U.S. Pat. No. 4,946,527 entitled “Pressure-Sensitive Adhesive Fastener and Method of Making the Same” issued to Battrell on Aug. 7, 1990. For simplicity, however, the fasteners will be described in terms of adhesive attachment means.

The adhesive attachment means are respectively covered by removable release liners, central pad release liner and garment facing adhesive release line (not shown). The adhesives should be covered with release liners to keep the adhesives from sticking to extraneous surfaces prior to use. Suitable release liners are described in U.S. Pat. No. 4,917,697.

The wings 112, 130 shown are integral with the main body portion 111. In such a case, the topsheet 102 forms one surface of both the wings 112, 130 and the main body portion 111, and the backsheet 104 may form the other surface of the same. In addition, the absorbent material of the feminine hygiene product 100 may extend into the wings 112, 130 to form a wing absorbent core, as described in greater detail in U.S. Pat. No. 4,917,697, or the wings 112, 130 may be free of absorbent material. Alternatively, the wings may be comprised of separate pieces of material which are attached to the main body portion 111, as described in EP 606 358 B1.

The wings 112, 130 are each associated with main body portion 111 along a juncture. This is typically a longitudinally-oriented (or “longitudinal”) juncture, such as lines of juncture 121. As used herein, the terms “juncture” (or “line of juncture”) refer to regions where the wings 112, 130 extend from or are joined to the main body portion 111. These regions can be any of various curved or straight lines, but they are not limited to lines. Thus, these regions can comprise flanges, strips, intermittent lines, and the like.

The wings 112, 130 have a proximal edge 123 adjacent the line of juncture 121. A distal edge or outer most edge portion 118 (FIG. 4A) is remote from the line of juncture 121. As shown in FIGS. 2 and 4A, each wing 112, 130 is divided into a first wing portion 120 and a second wing portion 124 spaced along the transverse wing axis 152, where the second wing portion 124 is outside the first wing portion 120 such that it is positioned more distal from the longitudinal centerline 134 than the first wing portion 120. Adhesive 126 is provided between the topsheet 102 and the backsheet 104 in the first wing portion 120. Mechanical bonding 128 (e.g., crimping) is provided between the topsheet 102 and the backsheet 104 in the second wing portion 124.

The feminine hygiene product 100 can be divided into a front area 105 and a rear area 107, the front and rear areas divided by the flap transverse centerline or transverse wing axis 152. The front area 105 refers to the portion of the feminine hygiene product 100 that is oriented towards the wearer's front when the feminine hygiene product 100 is worn. The rear area 107 refers to the portion of the feminine hygiene product 100 that is oriented towards the wearer's rear when the hygiene product 100 is worn.

The feminine hygiene product 100 can have a front end 125 and a rear end 127. The front end 125 and the rear end 127 are generally disposed along the principal longitudinal centerline 134. The front end 125 refers the end of the feminine hygiene product 100 that is oriented towards the wearer's front when the feminine hygiene product is worn. The rear end 127 refers to the portion of the feminine hygiene product 100 that is oriented towards the wearer's rear when the feminine hygiene product 100 is worn.

The overall size of the wings 112, 130 can be readily selected by those skilled in the art. Preferably, the wings 112, 130 are sized so that the feminine hygiene product 100 is from about 10 to about 23 centimeters wide between the outer most edge portions 118 of the wings 112, 130 at their greatest separation. Preferably each wing 112, 130 is from about 5 to at least about 19 centimeters long in the direction parallel to the principal longitudinal centerline 134 of the feminine hygiene product 100.

The shape of the wings 112, 130 can be selected by those skilled in the art. As shown in FIG. 2, the wings 112, 130 are asymmetrical about the longitudinal centerline 134. However, in other aspects, other feminine hygiene products formed with the method disclosed herein feature wings that are symmetrical about the longitudinal centerline 134. Thus, the aspects of the disclosure herein are not limited to wings that are either symmetrical or asymmetrical about the longitudinal centerline 134.

### Improved Wing Structure of the Feminine Hygiene Product

The aspects of the disclosure will now be discussed herein for the improved wing structure of the feminine hygiene product. FIG. 2 is an example according to various aspects illustrating a top view of a feminine hygiene product 100 with asymmetrical wings 112, 130 formed using the improved manufacturing process. As shown in FIG. 2, in one aspect the absorbent core 106 has a first longitudinal edge 108 and a second longitudinal edge 110. The first wing 112 extends laterally outward of the first longitudinal edge 108 and the second wing 130 extends laterally outward of the second longitudinal edge 110 (e.g., in a direction parallel to the transverse wing axis 152). As shown in FIG. 2, in some aspects of the feminine hygiene product 100 the leading edge portion 114 and the trailing edge portion 116 are asymmetrical about the transverse wing axis 152.

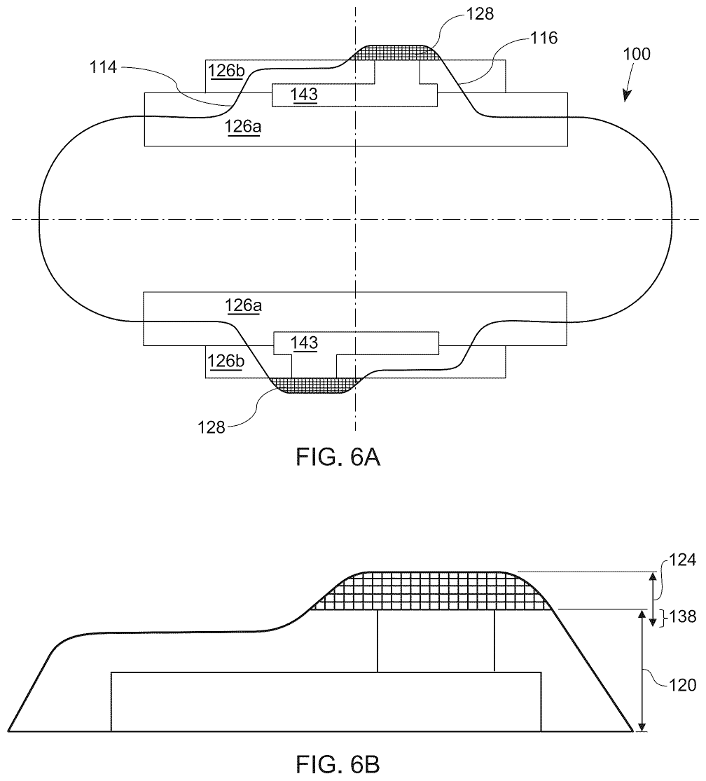

The improved feminine hygiene product disclosed herein is not limited to any specific wing configuration. In the example aspect of FIG. 2, the wings 112, 130 of the feminine hygiene product 100 are asymmetrical about the longitudinal centerline 134 of the feminine hygiene product 100. However, the aspects of the disclosure are not limited to this type of feminine hygiene product wing configuration and in other aspects (FIGS. 5A-5B) of the disclosure the method is used to manufacture feminine hygiene products 100′, 100″ where the respective wings (112′, 130′) (112″, 130″) are symmetrical with respect to the longitudinal centerline 134.

FIG. 3 is an example according to various aspects illustrating a cross-sectional view of the feminine hygiene product 100 taken along the line 3-3 in FIG. 2. As shown in FIG. 3, in one aspect the absorbent core 106 is disposed between the topsheet 102 and the backsheet 104. It is to be appreciated in other embodiments, the feminine hygiene product may also include a secondary backsheet disposed between the backsheet and the absorbent core and/or a secondary topsheet disposed between the topsheet and the absorbent core, such as disclosed in U.S. Pat. Nos. 10,532,123 and 11,389,336; and U.S. Patent Publication Nos. 2019/0099301; 2020/0306099; and 2020/0315870.

The wings of the improved feminine hygiene product 100 will now be discussed. FIG. 4A is an example according to various aspects illustrating a cross-sectional view of the feminine hygiene product 100 taken along the line 4-4 in FIG. 2. As shown in FIGS. 2 and 4A, in one aspect the wing 112 includes a first layer 113, a second layer 115, a leading edge portion 114, a trailing edge portion 116 and an outer most edge portion 118 joining the leading edge portion 114 and the trailing edge portion 116. In some aspects, the first layer 113 is a portion of the topsheet 102 and the second layer 115 is a portion of the backsheet 104. However, in other aspects the wing 112 includes the first and second layers 113, 115 which are different from the topsheet 102 and backsheet 104. The second wing 130 of the feminine hygiene product 100 is formed using the same process and thus has similar features as the first wing 112 disclosed herein.

A first wing portion of the wing will now be discussed. As shown in FIG. 4A, in some aspects the first wing 112 includes a first wing portion 120 where the first layer 113 and the second layer 115 are joined by the adhesive 126. For purposes of this description, the first wing portion 120 is defined as that portion of the wing 112 (or wing 130) where the adhesive 126 is provided between the first and second layers 113, 115. Additionally, as shown in FIG. 4A, the first wing portion 120 is defined as that portion of the first wing 112 that extends from the first longitudinal core edge 108 of the absorbent core 106 to a first edge 122. In this example aspect, the first edge 122 is defined as the edge of the adhesive 126 within the first wing 112.

A second wing portion of the wing will now be discussed. As shown in FIG. 4A, in some aspects the first wing 112 includes a second wing portion 124 that is laterally outward from the first wing portion 120 (along the transverse wing axis 152). In an example aspect, the second wing portion 124 is defined as that portion of the first wing 112 that extends from the first edge 122 (e.g., edge of the adhesive 126 within the first wing 112) to the outer most edge portion 118 of the first wing 112. In this aspect, in the second wing portion 124 the first layer 113 and the second layer 115 are joined by mechanical bonding 128 that extends to the outer most edge portion 118 of the first wing 112. FIG. 4A depicts a boundary 186 line between the first layer 113 and the second layer 115 within the second wing portion 124. In some aspects, no adhesive 126 is provided between the first and second layers 113, 115 in the second wing portion 124. In some example aspects, the mechanical bonding 128 includes at least one of crimping, ultrasonic bonding, pressure bonding, and thermal bonding.

In some example aspects, an overlap between the first wing portion 120 and the second wing portion 124 is present. As shown in FIG. 4A, an overlap 138 is provided between the first wing portion 120 and the second wing portion 124. The overlap 138 is defined as the extent to which the adhesive 126 extends into the second wing portion 124 with the mechanical bonding 128. As shown in FIG. 4A, in some aspects, the adhesive 126 extends within the overlap and terminates at an end 184 within the overlap 138 and thus does not extend into the second wing portion 124 beyond the overlap 138. In some aspects of the disclosure, the overlap 138 is adjusted to be greater than 0 mm, since this advantageously ensures that no portion of the wing 112 is unbonded. This also ensures the structural integrity of the wings 112, 130 as the feminine hygiene product 100 is being used. If there was no overlap 138, then it would be possible that in some portion of the wing 112 (e.g., between the mechanical bonding 128 and the adhesive 126) the first and second layers 113, 115 would not be bonded together.

Additionally, as previously discussed herein, the disclosure achieves the advantage of reducing waste by decreasing the width of the topsheet 102 or first layer 113 in this example aspect. Thus, based on this reduced or decreased width of the topsheet 102 there is a risk that the adhesive 126 could be exposed. In order to avoid this risk, the design of the first wing 112 is configured so that the width of the second wing portion 124 is greater than the overlap 138. This ensures that the first and second layers 113, 115 are mechanically bonded together for at least some portion of the wing 112 outside the area of the adhesive 126 and thus the adhesive 126 is not exposed. In one example aspect, the overlap 138 is greater than about 0 millimeters (mm) and less than about 2 mm whereas the width of the second wing portion 124 is between about 3 mm and about 9 mm. In one example aspect, the overlap 138 is about 1 mm.

Although FIG. 4A depicts that the first and second layers 113, 115 of the wing 112 have the same width and both extend to the outer most edge portion 118 of the wing 112, in other aspects the first and second layers 113, 115 have different widths such that they do not both extend to the outer most edge portion 118 of the wing 112. FIG. 4B is an example according to various aspects illustrating a cross-sectional view of the feminine hygiene product 100 taken along the line 4-4 in FIG. 2. As shown in FIG. 4B, in this example aspect the first layer 113 (e.g., topshect 102) has a reduced width as compared with the second layer 115 (e.g., backsheet 104). Thus, in this example aspect the second layer 115 (e.g., backsheet 104) forms the outer most edge portion 118 of the wing 112. Thus, in this example aspect a gap 148 is provided between the outer edge 149 of the first layer 113 (e.g., topsheet 102) and the outer most edge portion 118 of the wing 112, thereby exposing a portion of the second layer 115 (e.g., backsheet 104) over this gap 148. In aspects of the disclosure, the gap 148 is less than a width of the second wing portion 124 where the first and second layer 113, 115 are mechanically bonded (e.g., crimped) together. This ensures that the first and second layers 113, 115 are mechanically bonded (e.g., crimped) over at least some width of the wing 112 outside the adhesive 126 of the first wing portion 120 and thus prevent adhesive exposure. In an example aspect, the gap 148 is greater than about 0 mm and less than about 3 mm whereas the width of the second wing portion 124 is between about 3 mm and about 9 mm. In some aspects of the disclosure, setting the gap 148 and second wing portion 124 width in theses ranges ensures that even if the backsheet 104 is exposed (due to shifting and/or manufacturing tolerances) the gap 148 between the topsheet and backsheet 102, 104 is less than about 3 mm. This is based on the determination that a gap 148 greater than about 3 mm may be noticeable to the consumer. As appreciated by one skilled in the art, the backsheet 104 looks different than the topsheet 102 and thus this arrangement advantageously ensures that even if the gap 148 is present between the topsheet 102 and backsheet 104, such gap 148 is not visually noticeable to the consumer.

As shown in FIG. 4B, in one aspect the first wing 112 has a wearer facing surface 140 and a garment facing surface 142. The garment facing surface 152 comprises a garment fastening adhesive 143 which is used to be folded underneath the undergarment to secure the feminine hygiene product 100 to the underside of the undergarment. As shown in FIG. 4B, in some aspects of the disclosure a gap 144 is present between the garment facing adhesive 143 in the first wing portion 120 and the second wing portion 124. In an example aspect, the gap 144 is provided to ensure that the garment facing adhesive 143 does not interfere with the mechanical bonding 148 of the first and second layers 113, 115. In this example aspect, the gap 144 is in a range from about 1 mm to about 3 mm. However, in other aspects, no gap 144 is provided and thus the garment facing adhesive 143 need not be positioned within the first wing portion 120 at a certain minimum gap from the second wing portion 124 and may even be positioned within the second wing portion 124.

Although various aspects of the disclosure discuss that the first layer 113 of the first wing 112 is a portion of the topsheet 102 and the second layer 115 of the first wing 112 is a portion of the backsheet 104, in other aspects one or both of the first and second layers 113, 115 are distinct from the topsheet 102 and backsheet 104. In these aspects, the first and second layers 113, 115 are additional layers of material forming the first wing 112. In this example aspect, the first layer 113 and/or the second layers 115 are joined to the topsheet 102 and/or the backsheet 104.

Although certain aspects of the disclosure previously discussed involve forming the improved feminine hygiene product 100 with asymmetrical wings 112, 130 about the longitudinal centerline 134, in other aspects an improved feminine hygiene product is formed with symmetrical wings about the longitudinal centerline 134. FIGS. 5A and 5B are examples according to various aspects illustrating a top view of a feminine hygiene product 100′, 100″ with symmetrical wings formed using the improved manufacturing process. In one aspect, as shown in FIG. 5A the improved feminine hygiene product 100′ includes first and second wings 112′, 130′ that are symmetrical with respect to the longitudinal centerline 134. As further depicted in FIG. 5A, in this aspect of the disclosure the first wing 112′ includes the first wing portion 120′ and the second wing portion 124′. As with the first wing 112 of FIGS. 2 and 4A, the first wing portion 120′ features adhesive 126 provided between the first and second layers 113, 115 and the second wing portion 124′ features the mechanical bonding 128 between the first and second layers 113, 115. Due to the variation of the shape of the second wing portion 124′ (e.g., having a hub along an intermediate portion of the first wing 112′) the shape of the mechanical bonding 128 (e.g., crimped region) is different from the shape of the second wing portion 124 of FIGS. 2 and 4A.

In another aspect, as shown in FIG. 5B the improved feminine hygiene product 100″ includes first and second wings 112″, 130″ that are symmetrical with respect to the longitudinal centerline 134. As further depicted in FIG. 5B, in this aspect of the disclosure the first wing 112″ includes the first wing portion 120″ and the second wing portion 124″. As with the first wing 112 of FIGS. 2 and 4A, the first wing portion 120″ features adhesive 126 provided between the first and second layers 113, 115 and the second wing portion 124″ features the mechanical bonding 128 between the first and second layers 113, 115. Due to the variation of the shape of the second wing portion 124″ (e.g., having a hub along an intermediate portion of the first wing 112″ that is slightly deeper than the hub of the first wing 112′ of FIG. 5A) the shape of the mechanical bonding 128 (e.g., crimped region) is different from the shape of the second wing portion 124 of FIGS. 2 and 4A and the second wing portion 124′ of FIG. 5A.

The first and second wings of both of the improved feminine hygiene products 100′, 100″ share similar characteristics as the first and second wings 112, 130 of the improved feminine hygiene product 100 discussed herein.

It should be noted that the proposed approach would work with a different layout of the feminine hygiene product 100 and different wing design. Moreover, it would still be applicable even with different material used for the topsheet 102 and backsheet 104 and with different crimp patterns of the mechanical bonding 128 in the second wing portion 124.

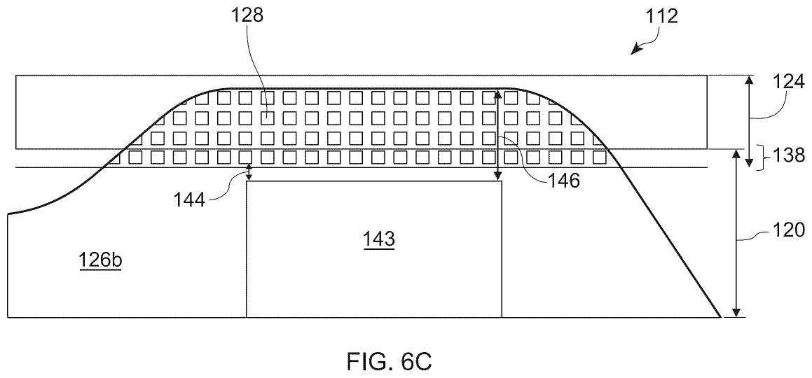

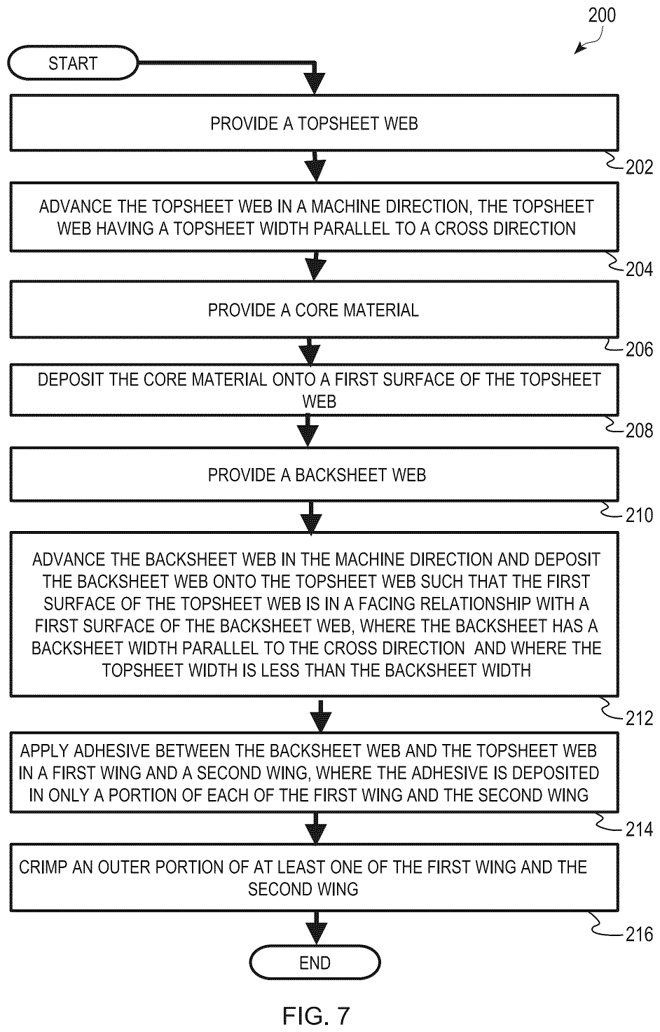

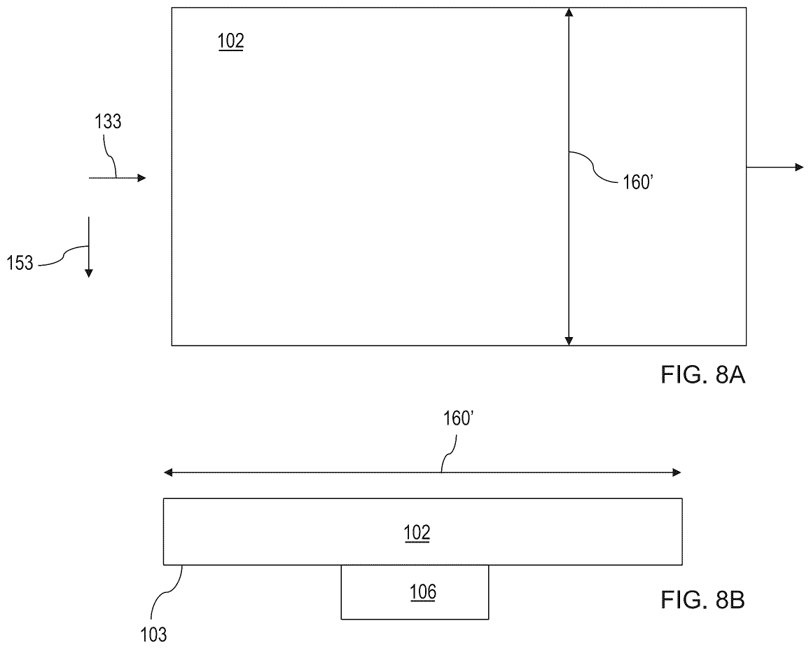

In addition to the side cross-sectional views of the wing design shown in FIGS. 4A and 4B, top-cross sectional views of the wing design will now be discussed which further describe the aspects of the improved wing design. FIG. 6A is an example according to various aspects illustrating a bottom view of the feminine hygiene product 100 of FIG. 2 with the adhesive layers 126 and mechanical bonding region 128 formed using the improved manufacturing process. In one aspect, as shown in FIG. 6A the adhesive 126 includes a first adhesive layer 126a that extends between the topsheet 102 and backsheet 104 in the main body portion 111 of the feminine hygiene product 100 and a second adhesive layer 126b that extends into the first and second wings 112, 130. In one example aspect, the second adhesive layer 126b is a wing laminate adhesive (WLA) since it is within the first and second wings 112, 130. As shown in FIG. 6A, in some aspects the second adhesive layer 126b extends from the leading edge portion 114 to the trailing edge portion 116 of the wings 112, 130. As further shown in FIG. 6A, in some aspects the garment facing adhesive 143 is depicted which is on the garment facing surface 142 of the wings 112, 130. In one example aspect, the garment facing adhesive 143 is a panty fastening adhesive (PFA) since it is typically adhered to an undersurface of a panty worn by the user of the feminine hygiene product 100. As illustrated, the panty fastening adhesive may be applied in various shapes.

FIG. 6B is an example according to various aspects illustrating a top view of the first wing 112 of the feminine hygiene product 100 of FIG. 6A including first and second wing portions 120, 124. As shown in FIG. 6B, the overlap 138 is depicted between the first and second wing portions 120, 124. As previously disclosed, the overlap 138 is in a range between about 0 mm and about 2 mm, such as about 1 mm, for example. In this example aspect, the 1 mm overlap 138 between the mechanical bonding 128 (e.g., crimped region) and the second adhesive layers 126b (e.g., WLA) is so that there is no gap in the bonding between the first layer 113 (e.g., topsheet 102) and the second layer 115 (e.g., backsheet 104) in the wing 112. However, in other example aspects there may be no overlap 138 or the overlap 138 may be greater than 2 mm, given process variability. As illustrated in FIG. 6B, the mechanical bonding 128 may include a bond pattern. The bond pattern may include a series of individual bonds that have a shape, such as a circle, oval, rectangle, square or any other bond shape. The bonds may be disposed in a uniform pattern, such as a grid patter as illustrated in FIG. 6B, or a non-uniform pattern. The bond pattern may also include substantially continuous bonds such that the first dimension of the bond is at least two times greater than the second dimension of the bond. For example, the first dimension extending substantially parallel to the longitudinal centerline 134 is at least two times greater than the second dimension extending substantially parallel to the transverse wing axis 152.

FIG. 6C is an example according to various aspects illustrating a top view of the first wing 112 of the feminine hygiene product 100 of FIG. 6A including the adhesive layers 126b and mechanical bonding region 128 in the first and second wing portions 120, 124. In one aspect, the gap 146 is depicted between the outer most edge portion 118 and the garment facing adhesive 143. In one example aspect, the gap 146 is at least about 3 mm or less than about 9 mm. It was recognized that in some example aspects, sizing the gap 146 within this range advantageously ensures that the garment facing adhesive 143 does not interfere with the mechanical bonding (e.g., crimping) of the second wing region 124. In some aspects of the disclosure, the width of the second wing portion 124 (e.g., measured along the transverse wing axis 152) is about 3 mm, about 6 mm or about 9 mm. However, the width of the second wing portion 124 is not limited to any particular value and can be selected within a range of values within or outside the range from about 3 mm to about 9 mm.

### Improved Manufacturing Process for Feminine Hygiene Product

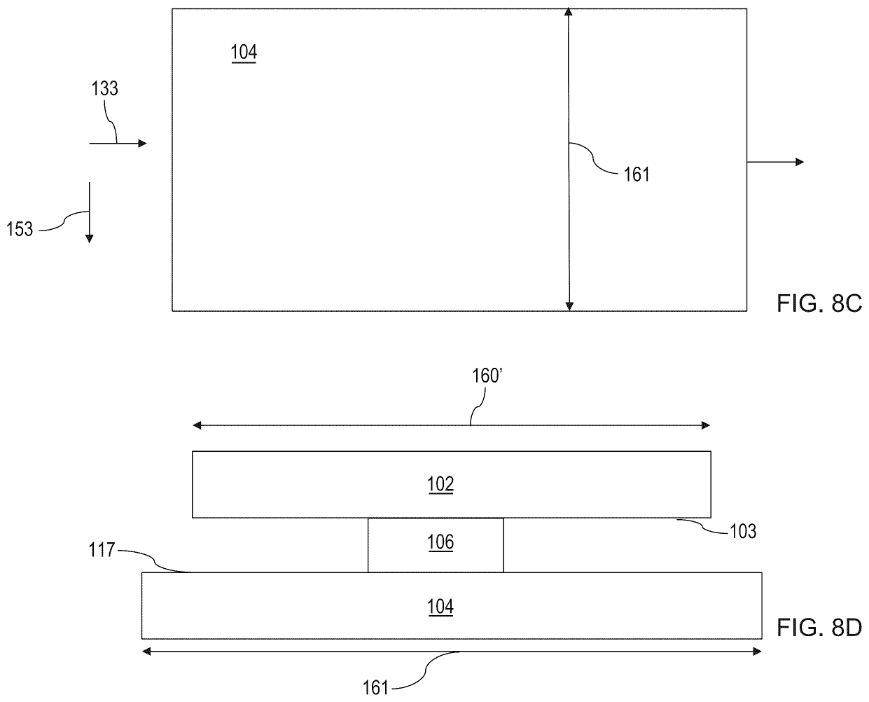

The method used to manufacture the improved feminine hygiene product 100 will now be discussed. FIG. 7 is an example according to various aspects illustrating a flow chart depicting steps of an improved method 200 for forming the feminine hygiene product 100 of FIG. 2. Although steps are shown in FIG. 7 as integral blocks in a particular order, in other aspects, one or more steps or portions thereof are performed in a different order or overlapping in time, in series or in parallel, or are omitted or one more additional steps are added.

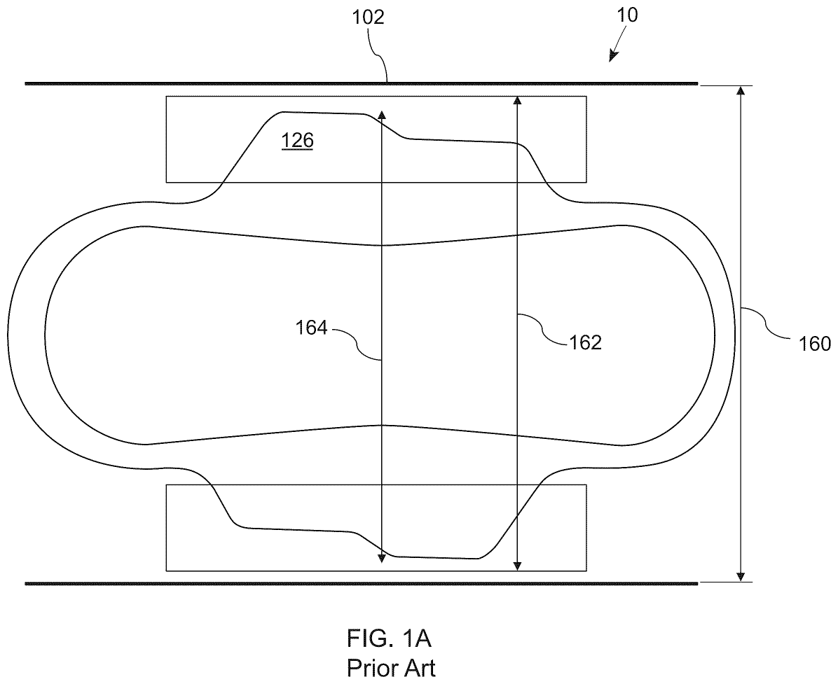

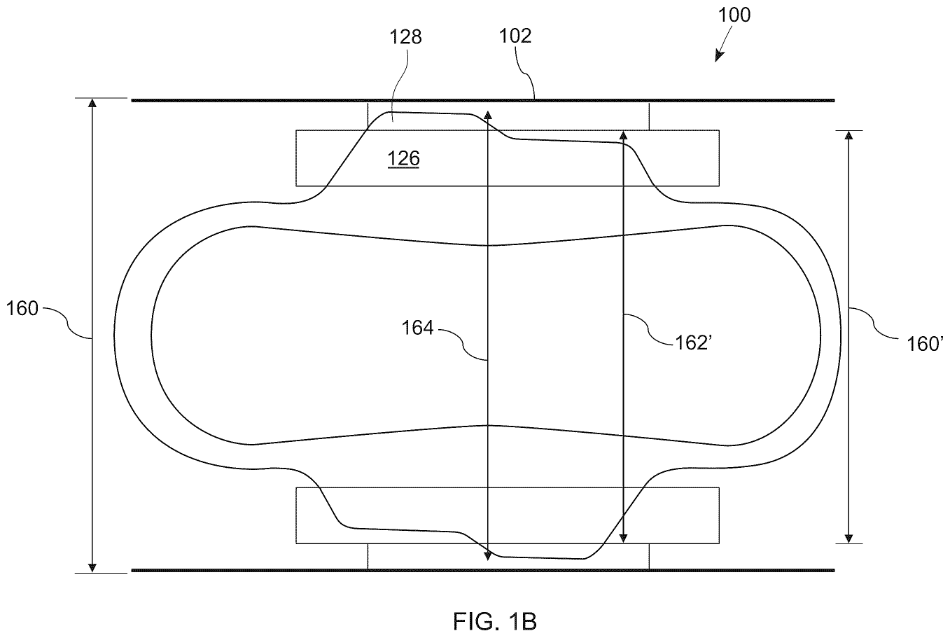

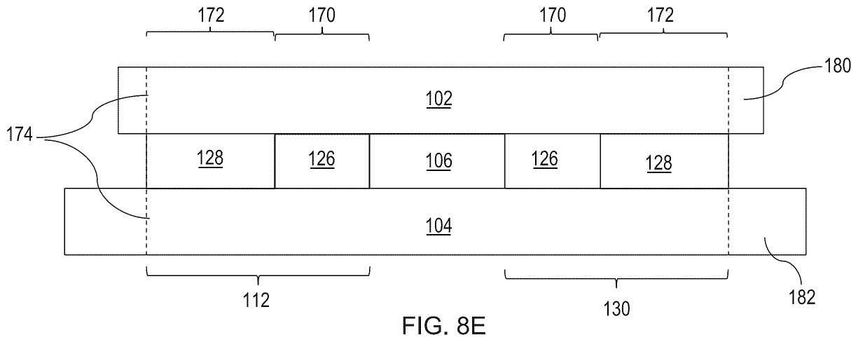

In step 202, the topsheet 102 web is provided. In step 204, the topsheet 102 web is advanced in the machine direction 133. FIG. 8A depicts one example aspect of the topsheet 102 web that has a width 160′ measured in a direction parallel to the cross direction 153 that is perpendicular to the machine direction 133. In one example aspect, the width 160′ of the topsheet 102 web is less than the width 160 of the topsheet 102 web used in conventional methods (FIG. 1A). In one example aspect, the width 160′ of the topsheet 102 web is about 160 mm whereas the width 160 of the topsheet 102 web used in the conventional method is about 168 mm.

In step 206, a core material 106 is provided. And in step 208, the core material 106 is deposited onto a first surface 103 of the topsheet 102 web material. FIG. 8B depicts one example aspect where the core material 106 is deposited onto the first surface 103 of the topsheet 102 web material. In other aspects, in step 208 the core material 106 can be deposited on a first surface 117 (FIG. 8D) of the backsheet 104.

In step 210, the backsheet 104 web is provided and in step 212 the backsheet 104 web is advanced in the machine direction 133. As shown in FIGS. 8C and 8D, in one aspect in step 212 the backsheet 104 web is deposited onto the topsheet 102 web such that the first surface 103 of the topsheet web is in a facing relationship with the first surface 117 of the backsheet 104 web. In some example aspects, the backsheet 104 web has a backsheet web width 161 measured in a direction parallel to the cross direction 153. In this example aspect, the topsheet width 160′ is less than the backsheet width 161. In one example aspect, the topsheet web width 160′ is less than the backsheet width 161 such that the difference between the topsheet width 160′ and the backsheet width 161 is from about 3 mm to about 15 mm or from about 5 mm to about 12 mm or from about 8 mm to about 10 mm or greater than about 3 mm or greater than about 5 mm or greater than about 8 mm or greater than about 10 mm.

In step 214, the adhesive 126 is applied between the backsheet 104 web and the topsheet 102 web in a first wing 112 and a second wing 130. As shown in FIG. 8E, in some aspects in step 214 the adhesive 126 is deposited in only a portion of each of the first wing 112 and the second wing 130. In one example aspect, in step 214 the adhesive 126 is deposited in an adhesive region 170 of the first and second wings 112, 130. In this example aspect, the adhesive region 170 corresponds to the first wing portion 120.

In step 216, an outer portion of at least one of the first wing 112 and second wing 130 is mechanically bonded. In one aspect, as shown in FIG. 8E, in step 216 the outer portion of the first wing 112 and/or second wing 130 is crimped such that the topsheet 102 web and backsheet 104 web are mechanically bonded together. In one example aspect, the outer portion of the first wing 112 and/or second wing 130 where step 216 is performed is in a crimped region 172 that corresponds to the second wing portion 124. Although FIG. 8E does not depict an overlap between the adhesive region 170 and the crimped region 172, this is merely for case of illustration and it is understood that the overlap 138 may be provided between the regions 170, 172 that is similar to the overlap 138 previously discussed herein. Additionally, for case of illustration the topsheet 102 and bottom sheet 104 in FIG. 8E are not shown to be crimped in the crimped region 172 but this is understood based on the disclosure herein. As previously discussed, the crimped region 172 may extend to the edge of the topsheet 102 and/or beyond the edge of the topsheet 102.

As appreciated by one of ordinary skill in the art, the reduction in the width 160′ of the topshect 102 as compared with the width 160 of the topsheet 102 used in conventional methods necessarily involves a decrease in the total width of the adhesive 126b (e.g., WLA) applied in step 214, in order to prevent adhesive exposure and contamination. Thus, in order to ensure closed wing edges, the aspects of the disclosure herein advantageously introduces step 216 for crimping the outer most edge portion 118. In one example aspect, the WLA mouthpiece applied in step 216 on the outer sides of the wing has been adjusted to apply a reduced width 162′ (e.g., about 148 mm) instead of the width 162 applied in conventional methods (e.g., about 160 mm). This reduces the adhesive 126b (outer WLA) application from 23 mm in the conventional method to about 17 mm in the disclosed method herein.

In an example aspect of the disclosure, various measurements and tests were conducted to evaluate different cut widths of the topsheet 102, adhesive 126b (e.g., WLA) widths, and crimp patterns of the crimped region 172. In these example aspects, the key parameters considered were the amount of exposed backsheet 104 web on the wings 112, 130; adhesive 126b (e.g., WLA) quality and dimensions; crimp width and quality on the wings 112, 130; topsheet 102 cross-direction position variation; topsheet 102 cut width roll variation; trim break occurrence analysis and functionality of the final knife. In these example aspects, after conducting several experimental observations and consumer tests, it was determined that an optimal configuration to align the process with product requirements is to use a topsheet width 160′ of about 160 mm, a total width 162′ of the adhesive 126b (e.g., WLA) of about 148 mm and a 6 mm crimp on the wings. However, the aspects of the disclosure are not limited to these particular numerical parameter values and include other values for these parameters outside these values.

As further shown in FIG. 8E, in another aspect the method includes a step of cutting the topsheet 102 web and the backsheet 104 web to form the feminine hygiene product 100. In one aspect, the cutting step involves cutting the perimeter of the feminine hygiene pad 100 shown in FIG. 2. In one example aspect, the cutting step is performed along cut lines 174 (FIG. 8E) to trim away excess top sheet 180 and excess bottom sheet 182 material that is not used to form the feminine hygiene product 100. The excess top sheet 180 that is trimmed away is far less than the equivalent amount of the increased width topsheet 102 that is cut away in the conventional method.

In some aspects of the disclosure, the feminine hygiene product 100 could be cut first and then crimped but this may be harder to manufacture. In one example aspect, a front and back end 125, 127 of the feminine hygiene product 100 are crimped at the same time as the wing is crimped in step 216. However, the crimping of the front and back end 125, 127 of the feminine hygiene product 100 can be done in two separate steps. The cutting step can then be performed after the crimping step.

It should be noted that in some aspects of the disclosure, reducing the topsheet 102 width 160′ increases the stiffness of the wing 112, 130 (due to the crimp) which provide a stiffer area for the user to grab to apply and remove the wing. It is believed that this stiffer, crimped wing area will provide a consumer benefit by allowing for greater control of the wing for application and removal.

### Combinations

In view of the foregoing disclosure, the following non-limiting examples are contemplated:
A. A feminine hygiene article comprising: a topsheet; a backsheet; an absorbent core disposed between the topsheet and the backsheet, wherein the absorbent core has a first longitudinal core edge and a second longitudinal core edge; and a first wing extending laterally outboard the first longitudinal core edge, wherein the first wing comprises a first layer, a second layer, a leading edge portion, a trailing edge portion and an outer most edge portion joining the leading edge portion and the trailing edge portion, the first wing has a first wing portion extending from the first longitudinal core edge to a first edge and a second wing portion extending from the first edge to the outer most edge portion, wherein the first layer and the second layer are joined by an adhesive in the first wing portion, and wherein the first layer and the second layer are joined by mechanical bonding in the second wing portion.
B. The feminine hygiene article according to paragraph A, comprising a second wing extending laterally outboard the second longitudinal core edge; wherein the first wing and the second wing are symmetrical about a longitudinal centerline of the feminine hygiene article.
C. The feminine hygiene article according to any one of the preceding paragraphs, comprising a second wing extending laterally outboard the second longitudinal core edge; wherein the first wing and the second wing are asymmetrical about a longitudinal centerline of the feminine hygiene article.
D. The feminine hygiene article according to any one of the preceding paragraphs, wherein an overlap is provided between the adhesive of the first wing portion and the mechanical bonding of the second wing portion.
E. The feminine hygiene article according to paragraph D, wherein the overlap is greater than 0 millimeters (mm) and less than 2 mm.
F. The feminine hygiene article according to paragraph E, wherein the overlap is about 1 mm.
G. The feminine hygiene article according to any one of the preceding paragraphs, wherein the first wing has a wearer facing surface and a garment facing surface, wherein the garment facing surface comprises a garment fastening adhesive and wherein a gap is present between the garment facing adhesive and the second wing portion.
H. The feminine hygiene article according to paragraph G, wherein a gap is provided between the outer most edge portion and the garment facing adhesive.
I. The feminine hygiene article according to paragraph H, wherein the gap is at least about 3 mm or less than about 9 mm.
J. The feminine hygiene article according to paragraph G, wherein the garment facing adhesive is disposed on the first wing portion.
K. The feminine hygiene article according to any one of the preceding paragraphs, wherein the first layer of the first wing is a portion of the topsheet.
L. The feminine hygiene article according to any one of the preceding paragraphs, wherein the second layer of the first wing is a portion of the backsheet.
M. The feminine hygiene article according to any one of the preceding paragraphs, wherein at least one of the first layer and the second layer of the first wing forms the outer most edge portion.
N. The feminine hygiene article according to any one of the preceding paragraphs, wherein the first layer forms a wearer facing surface of the first wing and the second layer forms a garment facing surface of the first wing; wherein the second layer forms the outer most edge portion of the first wing; and wherein there is a gap between the outer most edge portion and an outer edge of the first layer that is less than about 3 mm.
O. The feminine hygiene article according to any one of the preceding paragraphs, wherein the mechanical bonding includes at least one of crimping, ultrasonic bonding, pressure bonding, and thermal bonding.
P. The feminine hygiene article according to any one of the preceding paragraphs, wherein a width of the second wing portion is 3 mm, 6 mm, or 9 mm.
Q. The feminine hygiene article according to any one of the preceding paragraphs, wherein at least one of the first layer and the second layer of the first wing is joined to at least one of the topsheet and the backsheet.
R. The feminine hygiene article according to any one of the preceding paragraphs, wherein the adhesive extends from the leading edge portion to the trailing edge portion.
S. The feminine hygiene article according to any one of the preceding paragraphs, wherein the leading edge portion and the trailing edge portion are asymmetrical about a transverse wing axis.
T. A feminine hygiene article comprising: a topsheet; a backsheet; an absorbent core disposed between the topsheet and the backsheet, wherein the absorbent core has a first longitudinal core edge and a second longitudinal core edge; and a first wing extending laterally outboard the first longitudinal core edge, wherein the first wing comprises a portion of the topsheet, a portion of the backsheet, a leading edge portion, a trailing edge portion and an outer most edge portion joining the leading edge portion and the trailing edge portion, the first wing has a first wing portion extending from the first longitudinal core edge to a first edge and a second wing portion extending from the first edge to the outer most edge portion, wherein the first layer and the second layer are joined by an adhesive in the first wing portion, and wherein the first layer and the second layer are joined by mechanical bonding in the second wing portion; wherein an overlap is provided between the adhesive of the first wing portion and the mechanical bonding of the second wing portion, wherein the overlap is greater than 0 mm; and wherein the first wing has a wearer facing surface and a garment facing surface, wherein the garment facing surface comprises a garment fastening adhesive, wherein a gap is present between the garment facing adhesive and the second wing portion and wherein the gap is greater than 0 mm.
AA. A method of forming an absorbent article or feminine hygiene article, comprising: providing a topsheet web; advancing the topsheet web in a machine direction, wherein the topsheet web has a topsheet web width measured in a direction parallel to a cross direction that is perpendicular to the machine direction; providing a core material; depositing the core material onto a first surface of the topsheet web material; providing a backsheet web; advancing the backsheet web in the machine direction and depositing the backsheet web onto the topsheet web such that the first surface of the topsheet web is in a facing relationship with a first surface of the backsheet web, wherein the backsheet web has a backsheet web width measured in a direction parallel to the cross direction and wherein the topsheet web width is less than the backsheet web width; applying adhesive between the backsheet web and the topsheet web in a first wing and a second wing, wherein the adhesive is deposited in only a portion of each of the first wing and the second wing; and crimping an outer portion of at least one of the first wing and the second wing.
BB. The method according to paragraph AA, comprising forming a pad in the topsheet web and the backsheet web, wherein the pad comprises the first wing and the second wing formed from a portion of the topsheet web and the backsheet web.
CC. The method according to any one of the preceding paragraphs, comprising cutting the topsheet web and the backsheet web to form a pad, wherein the pad comprises the first wing and the second wing and at least one of the first wing and the second wing comprises an adhesive region and a crimped region.
DD. The method according to any one of the preceding paragraphs, wherein the topsheet web width is less than the backsheet web width by a difference that is in a range from about 3 mm to about 15 mm.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A feminine hygiene article comprising:
a topsheet;
a backsheet;
an absorbent core disposed between the topsheet and the backsheet, wherein the absorbent core has a first longitudinal core edge and a second longitudinal core edge; and
a first wing extending laterally outboard the first longitudinal core edge,
wherein the first wing comprises a first layer, a second layer, a leading edge portion, a trailing edge portion and an outer most edge portion joining the leading edge portion and the trailing edge portion,
the first wing has a first wing portion extending from the first longitudinal core edge to a first edge and a second wing portion extending from the first edge to the outer most edge portion,
wherein the first layer and the second layer are joined by an adhesive in the first wing portion, and
wherein the first layer and the second layer are joined by mechanical bonding in the second wing portion.

2. The feminine hygiene article according to claim 1, comprising a second wing extending laterally outboard the second longitudinal core edge;
wherein the first wing and the second wing are symmetrical about a longitudinal centerline of the feminine hygiene article.

3. The feminine hygiene article according to claim 1, comprising a second wing extending laterally outboard the second longitudinal core edge;
wherein the first wing and the second wing are asymmetrical about a longitudinal centerline of the feminine hygiene article.

4. The feminine hygiene article according to claim 1, wherein an overlap is provided between the adhesive of the first wing portion and the mechanical bonding of the second wing portion.

5. The feminine hygiene article according to claim 4, wherein the overlap is greater than 0 millimeters (mm) and less than 2 mm.

6. The feminine hygiene article according to claim 5, wherein the overlap is about 1 mm.

7. The feminine hygiene article according to claim 1, wherein the first wing has a wearer facing surface and a garment facing surface, wherein the garment facing surface comprises a garment fastening adhesive and wherein a gap is present between the garment facing adhesive and the second wing portion.

8. The feminine hygiene article according to claim 7, wherein a gap is provided between the outer most edge portion and the garment facing adhesive.

9. The feminine hygiene article according to claim 8, wherein the gap is at least about 3 mm or less than about 9 mm.

10. The feminine hygiene article according to claim 7, wherein the garment facing adhesive is disposed on the first wing portion.

11. The feminine hygiene article according to claim 1, wherein the first layer of the first wing is a portion of the topsheet.

12. The feminine hygiene article according to claim 1, wherein the second layer of the first wing is a portion of the backsheet.

13. The feminine hygiene article according to claim 1, wherein at least one of the first layer and the second layer of the first wing forms the outer most edge portion.

14. The feminine hygiene article according to claim 1, wherein the first layer forms a wearer facing surface of the first wing and the second layer forms a garment facing surface of the first wing;
wherein the second layer forms the outer most edge portion of the first wing; and
wherein there is a gap between the outer most edge portion and an outer edge of the first layer that is less than about 3 mm.

15. The feminine hygiene article according to claim 1, wherein the mechanical bonding includes at least one of crimping, ultrasonic bonding, pressure bonding, and thermal bonding.

16. The feminine hygiene article according to claim 1, wherein at least one of the first layer and the second layer of the first wing is joined to at least one of the topsheet and the backsheet.

17. The feminine hygiene article according to claim 1, wherein the adhesive extends from the leading edge portion to the trailing edge portion.

18. The feminine hygiene article according to claim 1, wherein the leading edge portion and the trailing edge portion are asymmetrical about a transverse wing axis.

19. A feminine hygiene article comprising:
a topsheet;
a backsheet;
an absorbent core disposed between the topsheet and the backsheet, wherein the absorbent core has a first longitudinal core edge and a second longitudinal core edge; and
a first wing extending laterally outboard the first longitudinal core edge,
wherein the first wing comprises a portion of the topsheet, a portion of the backsheet, a leading edge portion, a trailing edge portion and an outer most edge portion joining the leading edge portion and the trailing edge portion,
the first wing has a first wing portion extending from the first longitudinal core edge to a first edge and a second wing portion extending from the first edge to the outer most edge portion,
wherein the first layer and the second layer are joined by an adhesive in the first wing portion, and
wherein the first layer and the second layer are joined by mechanical bonding in the second wing portion;
wherein an overlap is provided between the adhesive of the first wing portion and the mechanical bonding of the second wing portion, wherein the overlap is greater than 0 mm; and
wherein the first wing has a wearer facing surface and a garment facing surface, wherein the garment facing surface comprises a garment fastening adhesive, wherein a gap is present between the garment facing adhesive and the second wing portion and wherein the gap is greater than 0 mm.

20. A method of forming an absorbent article or feminine hygiene article, comprising:
providing a topsheet web;
advancing the topsheet web in a machine direction, wherein the topsheet web has a topsheet web width measured in a direction parallel to a cross direction that is perpendicular to the machine direction;
providing a core material;
the core material onto a first surface of the topsheet web material;
providing a backsheet web;
advancing the backsheet web in the machine direction and depositing the backsheet web onto the topsheet web such that the first surface of the topsheet web is in a facing relationship with a first surface of the backsheet web, wherein the backsheet web has a backsheet web width measured in a direction parallel to the cross direction and wherein the topsheet web width is less than the backsheet web width;
applying adhesive between the backsheet web and the topsheet web in a first wing and a second wing, wherein the adhesive is deposited in only a portion of each of the first wing and the second wing; and
crimping an outer portion of at least one of the first wing and the second wing.
